(19) United States
(12) Patent Application Publication
(10) Pub. No.: US 2004/0076695 A1
(43) Pub. Date: Apr. 22, 2004

(54) EPA and DHA enriched omega-3 supplement for the treatment of dry eye, meibomianitis and xerostomia

(75) Inventor: Jeffrey P. Gilbard, Weston, MA (US)

Correspondence Address: LAHIVE & COCKFIELD, LLP., 28 STATE STREET, BOSTON, MA, 02109, US

(73) Assignee: ADVANCED VISION RESEARCH, Woburn, MA

(21) Appl. No.: 10/615,158

(22) Filed: Jul. 7, 2003

Related U.S. Application Data
(60) Provisional application No. 60/461,911, filed on Apr. 10, 2003.
(60) Provisional application No. 60/416,322, filed on Oct. 4, 2002.
(60) Provisional application No. 60/394,417, filed on Jul. 8, 2002.

(51) Int. Cl. A61K 35/78; A61K 31/225

(52) U.S. Cl. 424/765; 424/776; 514/547

(57) ABSTRACT

Nutritional supplements for treating dry eye, meibomian gland inflammation, meibomian gland dysfunction or xerostomia comprising flaxseed oil, or a GLA-rich oil, combined with a n-3 rich oil, such as fish oil which is rich in eicosapentaenoic acid (EPA) and docosahexaeonic acid (DHA), are disclosed. Methods of treating dry eye, meibomian gland inflammation, meibomian gland dysfunction or dry mouth by administering the supplements are also described.

### RELATED APPLICATIONS

This application claims priority to U.S. Serial No. 60/394417, filed on Jul. 8, 2002, entitled “Omega-3 Supplement for the Treatment of Dry Eye,” U.S. Serial No. 60/416322, filed on Oct. 4, 2002, entitled “EPA-Enriched Omega-3 Supplement for the Treatment of Dry Eye,” and U.S. Serial No. 60/461911, filed on Apr. 10, 2003, entitled “EPA and DHA Enriched Omega-3 Supplement for the Treatment of Dry Eye,” the contents of which are incorporated herein in their entirety by this reference.

### BACKGROUND OF THE INVENTION

Evaporative dry eye results from inflammation and dysfunction of the oil glands, or meibomian glands, in the eyelid. The oil produced by these glands coats the tear film of the eye. Dry eye from decreased aqueous tear production results from any condition that damages or decreases the function of the lacrimal glands, or any condition that decreases corneal sensation.

The normal tear film consists of three layers. The outer oil layer reduces evaporation of the remaining layers of tears, the middle aqueous layer provides electrolytes and proteins, and the inner mucous layer, which has direct contact with the eye surface, provides lubrication and helps to keep the aqueous layer on the surface of the eye. A deficiency of any or all of the three-layered tear film leads to dry eye, which results in irritation and damage to the surface of the eye.

Dietary intake of omega-3 essential fatty acids influence the polar lipid profile of meibomian gland secretions. (Sullivan et al. Third Intentional Conference on the Lacrimal Gland, Tear Film and Dry Eye Syndrome: Basic Science and Clinical Relevance, Maui, Hi., Nov. 15-18, 2000). In addition, dietary intake of both EPA and DHA effect the profile of the polar lipid fraction of the oils produced by the meibomian glands (Sullivan et al. Correlations between nutrient intake and the polar lipid profiles of meibomian gland secretions in women with Sjögren's syndrome. Lacrimal Gland, Tear Film, and Dry Eye Syndromes 3. Edited by D. Sullivan et al., Kluwer Academic/Plenum Publishers, 2002). These fatty acids contribute to the oil layer in the tear film, providing the raw materials for the production of meibomian gland oil that can properly exit the gland and coat the tear film. The omega-3 essential fatty acids can also decrease inflammation of the meibomian glands by generating anti-inflammatory mediators and decreasing inflammatory mediators. For example, a n-3 fatty acid such as eicosapentaenoic acid (EPA) can be converted into anti-inflammatory mediators prostaglandin E3 (PGE3) and leukotriene B5 (LTB5) which act to decrease inflammation. Dietary administration of fish oil containing EPA has produced a dose-dependent reduction of pro-inflammatory cytokines TNF-α, IL-1β, IL-1α, and cyclooxygenase 2 (COX-2). (Caughey et al. (1996) Am. J. Clin. Nutr. 63:116-122; Curtis et al. (2000) J. Biol. Chem. 275:721-724). The n-3 fatty acids, like EPA and DHA, decrease inflammation by promoting the conversion of the n-6 fatty acids to the Series 1 prostaglandins and inhibiting their conversion to the pro-inflammatory arachadonic acid (AA) pathway.

The consumption of omega-6 essential fatty acids (EFAs) far exceeds the consumption of omega-3 EFAs in the Western diet. Linoleic acid (C₁₈ n-6) is the root omega-6 and can be converted to either the Series 1 or Series 2 prostaglandins. Since the Series 2 prostaglandins are pro-inflammatory agents, there have been some attempts at modifying the fat content of the diet to treat meibomian gland inflammation (also known as meibomianitis or blepharitis), meibomian gland dysfunction, and dry eye. For example, flaxseed oil, a mix of n-6 and n-3 fatty acids, has been tried with some success. (Boerner, C. F. Dry eye successfully treated with oral flaxseed oil OSN, Oct. 15, 2000).

Accordingly, an object of the invention is to provide a nutritional supplement comprising a combination of selected omega-3 and omega-6 fatty acids for the treatment of dry eye, meibomian gland inflammation, xerostomia (also known as dry mouth) or meibomian gland dysfunction, e.g., a nutritional supplement which is better than the flaxseed oil alone. Another object of the invention is to provide a method of treating dry eye by administering such a nutritional supplement. Another object of the invention is to provide a method of treating meibomian gland inflammation or dysfunction or xerostomia by administering such a nutritional supplement.

### SUMMARY OF THE INVENTION

The present invention provides nutritional supplements for treating and preventing dry eye, meibomian gland inflammation (meibomianitis or blepharitis) or meibomian gland dysfunction, as well as methods for treating dry eye, meibomian gland inflammation or meibomian gland dysfunction by administering the supplements. The present invention also provides nutritional supplements for treating dry mouth, as well as a method for treating dry mouth. The supplements include a combination of selected n-3 and n-6 fatty acids. In particular, the nutritional supplements contain a source of n-6 fatty acids and a n-3 rich oil, wherein the n-3 rich oil contains a high concentration of eicosapentaenoic acid (EPA) and a high concentration of docosahexaeonic acid (DHA). The n-6 fatty acid-containing oil can further include a source of n-3 fatty acids. The n-6 fatty acid-containing oils are administered in nutritionally sufficient amounts and include, for example, flaxseed oil and gamma-linolenic (GLA) -rich oils such as evening primrose oil, borage oil, and black currant seed oil. Another source of a n-6 fatty acid includes dihomo-gamma linolenic acid (DGLA) either in natural or concentrated form. The nutritional supplements can also include a combination of flaxseed oil and an additional n-6 source.

EPA and DHA are easily found in very high concentrations in fish oils, primarily cold water fish oil, e.g., salmon, mackerel, sardines, herring, anchovies, rainbow trout, bluefish, caviar, and white albacore tuna canned in water. By using concentrated fish oil, or fish oil having a high concentration of EPA and DHA, the best results are achieved. For example, the preferred oil source for the n-3 fatty acids is a blend of n-3 rich oils, such as a fish oil, with one having at least about 40%-50% EPA, preferably, about 45% EPA, and the other having at least about 40%-50% DHA, preferably, about 50% DHA. Such oil blends are combined to produce a therapeutic amount of EPA and DHA for treating various conditions.

The nutritional supplements of the present invention can also include an oil soluble antioxidant, e.g., any form of vitamin E, preferably alpha-tocopherol. Other oil soluble antioxidants can include, among others, oryzanol and alpha-lipoic acid. Additional mixed tocopherols can also be included. In addition to vitamin E, the nutritional supplements can also include an amount of mixed tocopherols. Such a combination provides anti-inflammatory properties, as well as antioxidation properties. Preferably, the supplements contain approximately 100-400 IU of vitamin E, most preferably about 200 IU of vitamin E, and approximately 5-20 mg of mixed tocopherols, most preferably about 10 mg of mixed tocopherols, for approximately each 1.0 g of the n-6 fatty acid-containing oil, e.g., flaxseed and/or a GLA-rich oil, which is mixed with the appropriate amount of the n-3 rich oil, e.g., a high EPA and DHA fish oil to achieve the daily dose. The ratio of the n-6 fatty acid-containing oil to the n-3 rich oil can also vary. The ratios of the n-6 fatty acid-containing oil to n-3 rich oil range from about 25% to 75% (1 to 3) to about 75% to 25% (3 to 1). Ranges intermediate to the above-recited values, e.g., about 30% to 70%, about 60% to 40%, and about 50% to 50% are also intended to be encompassed by the present invention. Accordingly, the preferred daily dosage comprises the amount of the preferred EPA- and DHA-enriched n-3 rich oil or oils to provide approximately 150-550 mg of EPA, more preferably about 350-450 mg of EPA, and approximately 50-500 mg of DHA, more preferably about 250-350 mg of DHA.

The invention also features methods of treating a patient suffering from dry eye, meibomian gland inflammation (e.g., meibomianitis or blepharitis), meibomian gland dysfunction or xerostomia by administering orally the nutritional supplements of the present invention. Preferably, the daily dose of the supplement is administered once in the morning but it can be administered twice daily. For patients also afflicted with autoimmune diseases, e.g., Sjögren's syndrome or rheumatoid arthritis, twice the preferred daily dosage is recommended.

Other features and advantages of the instant invention will be apparent from the following detailed description and claims.

### BRIEF DESCRIPTION OF THE DRAWINGS

FIG. 1 is a flow chart showing the n-3 fatty acid pathway.

FIG. 2 is a flow chart showing the n-6 fatty acid pathway.

### DETAILED DESCRIPTION OF THE INVENTION

The present invention provides novel nutritional supplements for the treatment of dry eye, meibomian gland inflammation, meibomian gland dysfunction or dry mouth, as well as methods for administering such supplements. The supplements of the invention employ a combination of selected fatty acids to achieve n-3 and n-6 fatty acid mixes that are useful in the treatment of these symptoms.

Preferred combinations include oils that contain n-6 fatty acids and n-3 fatty acids rich in EPA and DHA. The n-6 fatty acid-containing oil can be selected to be an oil that includes n-3 fatty acids as well. Examples of n-6 oils capable of providing nutritionally sufficient amounts of a n-6 fatty acid include flaxseed oil and GLA-rich oils, such as evening primrose oil, borage oil, and black currant seed oil. Other sources of n-6 fatty acids contain GLA or DGLA, either in natural or concentrated form. The nutritional supplements can also include a combination of flaxseed oil and an additional source of n-6 fatty acids. Examples of n-3 oils rich in EPA and DHA and, therefore, capable of providing therapeutic amounts of EPA and DHA, include fish oils, primarily cold water fish oil, e.g., salmon, mackerel, sardines, herring, anchovies, rainbow trout, bluefish, caviar, and white albacore tuna canned in water.

As used herein, the term “n-3 rich oil” is a n-3 fatty acid containing oil having a high concentration of EPA and a high concentration of DHA. Such combinations of EPA and DHA can be achieved by using either natural or blended oils, e.g., a blend of oil rich in EPA and a blend of oil rich in DHA. In addition, EPA and DHA, as well as n-6 oils, are commercially available. The supplements function to relieve or prevent the symptoms associated with dry eye, meibomian gland inflammation, meibomian gland dysfunction or dry mouth.

As used herein, the terms “n-6 fatty acid-containing oil,” and “oil containing a n-6 fatty acid” are used interchangeably and include any compound which contains a n-6 fatty acid such as linoleic acid (LA) or GLA. Examples of such n-6 fatty acid-containing oils include, for example, flaxseed oil, and GLA-rich oils. Another source of a n-6 fatty acid includes DGLA, either in natural or concentrated form.

As used herein, the term “GLA-rich oil” includes all oils that contain a high concentration of GLA, e.g., about 9-30% or more GLA by weight. Examples of GLA-rich oils include evening primrose oil (approximately 9% GLA by weight), borage oil (approximately 25% by weight), and black currant seed oil (approximately 15% GLA by weight).

As used herein, the term “high concentration of EPA” is defined as a n-3 oil containing at least about 150-550 mg of EPA in 0.5-1.5 g of the n-3 rich oil and, preferably, about 450-500 mg of EPA in 1.4-1.5 g of the n-3 rich oil. Similarly, the term “high concentration of DHA” is defined as a n-3 oil containing at least about 50-500 mg of DHA in 0.5-1.5 g of the n-3 rich oil and, preferably, about 250-500 mg of DHA in 1.4-1.5 g of the n-3 rich oil.

As used herein, the term “a nutritionally sufficient amount” includes the amount of n-6 fatty acids required to satisfy the nutritional needs of a subject. This amount of n-6 fatty acids is helpful in treating a variety of conditions, i.e., relieving or reducing the symptoms associated with a particular condition, such as dry eye, meibomian gland inflammation, meibomian gland dysfunction, or dry mouth.

The term “a therapeutic amount” includes the amount of a n-3 and n-6 fatty acid which is capable of treating conditions, i.e., capable of relieving or reducing the symptoms associated with a particular condition, such as dry eye, meibomian gland inflammation, meibomian gland dysfunction, or dry mouth.

As used herein, the term “fatty acids” is art recognized and includes a long-chain hydrocarbon based carboxylic acid. Lipids are long chain polyunsaturated fatty acids which can be classified into three major groups: omega-3 (“n-3”), omega-6 (“n-6”), and omega-9 (“n-9”). The classes are based on the location of the double bond closest to the methyl end of the fatty acid; that is, if the closest double bond is between the third and fourth carbon atoms from the methyl group, the molecules are n-3 fatty acids, while if the double bond is between the sixth and seventh carbon atoms, the molecules are classified as n-6 fatty acids. Man and other mammals can desaturate or elongate the fatty acid chains but cannot interconvert fatty acids from one family to another. Although most of the fatty acids consumed in normal nutrition have sixteen (C₁₆) or eighteen carbon (C₁₈) chains, the twenty or greater carbon fatty acids, whether ingested or made in the body, are the most important in terms of physiological functions. The n-9 fatty acids are primarily elongated to form the twenty carbon eicosatrienoic (C₂₀:3 n-9) while the most important twenty carbon n-6 fatty acid is arachidonic acid (C₂₀:4 n-6). The n-3 fatty acids are normally elongated and desaturated to form either the twenty carbon eicosapentaenoic (C₂₀:₅n-3) or the twenty-two carbon docosahexaenoic (C₂₂:₆n-3). The notation (C_:_{—n-}_) indicates the number of carbon atoms in the chain, the number of double bonds, and the class of the fatty acid, respectively.

One of the reasons why the twenty carbon, or greater, fatty acids are important is their ability to act as substrates in the various prostanoid synthesis pathways, the chemical reactions which form prostaglandins from fatty acids. Prostaglandins, thrombozanes, leukotrienes, and lipoxins are localized tissue hormones that are fundamental regulating molecules in most forms of life. Prostaglandins are produced in the cells by the action of enzymes on essential fatty acids. There are distinct prostaglandin pathways for each class of fatty acids, one that begins with double-unsaturated n-6 linoleic acid and one that begins with triple-unsaturated n-3 alpha-linolenic acid. Each pathway involves elongation of the 18-carbon fatty acid to the 20-carbon root used in each of the three eicosanoid types, plus further desaturation.

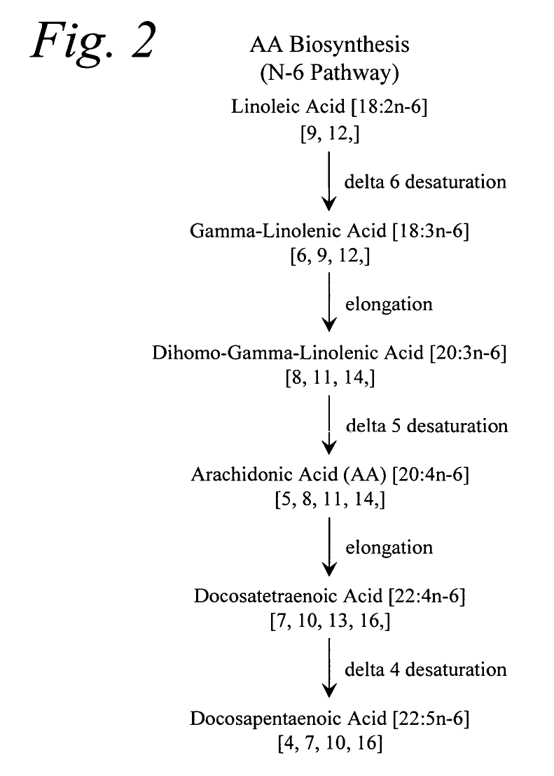

As shown in FIG. 2, the n-6 pathway begins with double-unsaturated linoleic acid (LA). This is one of the primary dietary fatty acids in the western diet, and is found in seed oils, e.g., flaxseed oil. LA is desaturated by the action of a desaturating enzyme, delta-6 desaturase (D6D), resulting in an 18-carbon, triple-unsaturated fatty acid, GLA. Two more carbon atoms are added to GLA by an elongase enzyme to form a 20-carbon triple-unsaturated fatty acid, DGLA, which is also found in liver and other organ meats. DGLA forms the root of the Series 1 prostaglandins such as PGE₁, PGF₁ₐ, and PGD₁, and thromboxanes such as TXA₁.

DGLA can also be transformed into 20-carbon quadruple-unsaturated arachidonic acid (AA), which is the root or precursor of the Series 2 eicosanoids and which is also found in butter, animal fats, especially pork, organ meats, eggs and seaweed. The Series 2 family includes a number of prostaglandins such as PGE₂, PGF₂ₐ and PGD₂, prostacyclins such as PGI₂, thromboxanes such as TXA₂, leukotrienes and lipoxins which are formed when AA interacts with the enzyme cyclooxygenase. Series 2 prostaglandins promote swelling, inflammation, and clotting, while Series 1 prostaglandins have the opposite effect.

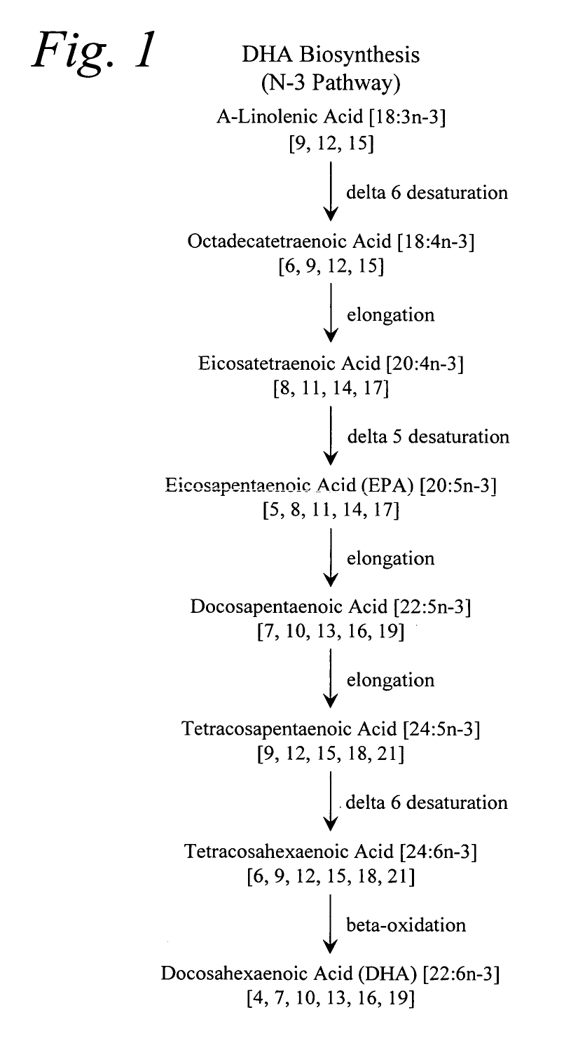

While AA is the most prominent member of the n-6 pathway, EPA and DHA are the most prominent members of the n-3 pathway. As shown in FIG. 1, these fatty acids are the elongation and desaturation products of the essential fatty acid, alpha-linolenic acid (ALA). ALA is found in seed oils of northern origin, like flax. This essential fatty acid is desaturated twice and elongated once to produce EPA, a 20-carbon fatty acid with five double bonds which is found plentifully in fish oils, e.g., menhaden, and fish eggs. EPA is the root substance of the Series 3 family that includes the prostaglandins such as PGE₃, PGH₃ and PGI₃, and thromboxanes such as TXA₃. EPA is then further elongated and desaturated to produce docosahexaeonic acid (DHA), a 22-carbon fatty acid with six double bonds. DHA is found plentifully in the brain and is in fact essential for the development and function of the brain. DHA also acts as a storage molecule. It can be shortened and resaturated to produce EPA and the Series 3 prostaglandins.

The n-⁶ and n-3 pathways are independent from each other. However, each compete for the same elongation and desaturation enzymes and for the site of esterification at the 2 position of the lipids. Accordingly, since both n-3 and n-6 fatty acids can be used as substrates for the prostaglandin pathways, it is possible to modify the results of these pathways by modifying the dietary intake of n-3 and n-6 fatty acids.

### Modifications Caused by n-3 Fatty Acids Rich in EPA

Increasing the amount of n-6 fatty acids alone, such as by adding a GLA-rich oil, increases the amount of DGLA produced and the amount of AA, as well as the pro-inflammatory metabolites associated with AA. This is counterproductive in the treatment of dry-eye. However, by adding a n-3 fatty acid which contains a high concentration of EPA, the EPA competitively inhibits conversion of DGLA to AA, thus, promoting the synthesis of PGE₁. PGE₁ is anti-inflammatory suppressing meibomianitis. Further, the addition of a n-6 fatty acid containing oil, e.g., GLA, increases the amount of substrate available for interaction with EPA and, accordingly, results in the production of more PGE₁. In turn, PGE₁ binds to EP2 and EP4 receptors to activate adenylate cyclase and increase cyclic adenosine monophosphate (cAMP) which is known to stimulate aqueous tear production, and salivary secretion. In addition, increasing n-3's, via increasing EPA, increases the production of PGE2 and LTB5, both of which are anti-inflammatory, further suppressing meibomian gland inflammation. High EPA concentrations in the nutritional supplements also serve to decrease the gene expression of proteoglycan degrading enzymes (aggrecanases), and pro-inflammatory IL-1β, IL-1α, tumor necrosis factor-α (TNF-α), and cyclooxygenase 2 (COX-2). Finally, omega-3 supplementation, such as EPA- and DHA-supplementation, modifies the lipid profile of the meibomian gland secretions. In these ways the nutritional supplements treat meibomian gland inflammation, meibomian gland dysfunction, dry eye and dry mouth.

Increasing the n-3 fatty acid, e.g., EPA, also inhibits the AA inflammatory cascade. Therefore, as indicated earlier, higher concentrations of EPA decrease the production of pro-inflammatory mediators.

In addition, high EPA concentrations can block lacrimal gland and corneal and conjunctival apoptosis (programmed cell death) by blocking the gene expression of TNF-α. Specifically, it is known that TNF-α upregulates apoptosis in salivary duct epithelial cells in human salivary ducts. Accordingly, TNF-α which is secreted by infiltrating lymphocytes induces apoptosis of the salivary gland in patients afflicted with Sjögren's syndrome. (Matsumura R. et al. (2000) Clin Exp Rheumatol 18(3):311-8). Since EPA is known to block TNF-α gene transcription, EPA, i.e., high concentrations of EPA in the nutritional supplements of the present invention, block or inhibit apoptosis in the lacrimal gland, the corneal and conjunctival epithelium and the salivary gland, thereby blocking or inhibiting lacrimal gland, corneal and conjunctival, and salivary gland apoptosis. This further contributes to the supplement's efficacy in treating or preventing dry eye and dry mouth.

### Modifications Caused by n-3 Fatty Acids Rich in DHA

DHA has been found to correlate inversely with dry-eye disease activity in Sjögren's syndrome (Oxholm et al. (1998) Prostaglandins, Leukotrienes and Essential Fatty Acids 59(4):239-45). Specifically, there is a significant inverse correlation between DHA levels in cell membranes, e.g., erythrocyte phospholipids, plasma phospholipids, plasma phospholipids and plasma triglycerides, and surface exocrine disease activity, i.e., eye, mouth, nasal, laryngotracheal, pharyngooesophageal, ,and lacrimal and salivary gland disease. Therefore, DHA is an important supplement in the prevention or treatment of dry eye syndrome, and dry mouth

DHA has also been found to inhibit cell apoptosis (Akbar et al. (2002) J. Neurochem. 2002 Aug;82(3):655-665; and Kishida et al. (1998) Biochim. Biophys. Acta 1391(3):401-8; Yano et al. (2000) J. Nutr. 130(5):1095-101). Accordingly, it is likely that DHA can block apoptosis of lacrimal gland secretory cells and salivary gland secretory cells, thereby decreasing the autoimmune destruction of the lacrimal gland and salivary glands which occurs in Sjögren's syndrome and other disorders similarly effecting the lacrimal glands of the eye or the salivary glands of the mouth. Accordingly, the nutritional supplements of the present invention can treat dry eye by protecting and preserving lacrimal gland function, and dry mouth by protecting and preserving salivary gland function.

In addition, DHA is responsible for thinning the oils so that they exit the meibomian glands more easily, thereby decreasing the stasis that helps to promote meibomianitis. Thinner oils also function to better coat the tear film, thereby retarding evaporation and diminishing dry eye.

Accordingly, the nutritional supplements of the present invention can treat meibomianitis and contribute to the improvement in function of the meibomian glands, thereby treating dry eye.

The nutritional supplements of the present invention can further include an oil soluble antioxidant, e.g., any form of vitamin E, preferably d-alpha-tocopherol. Other oil soluble antioxidants include, among others, oryzanol and alpha-lipoic acid. Additional mixed tocopherols can also be included. As part of the nutritional supplements of the present invention, vitamin E works to prevent the oxidation of the n-3 fatty acids, while also preventing the depletion of systemic vitamin E levels in the patient. In addition, vitamin E works synergistically with DHA to inhibit TNFα-induced apoptosis. Accordingly, a high concentration of vitamin E is preferred, e.g., at least about 150-250 IU of vitamin E, preferably about 200 IU of vitamin E plus 10-20 mg of mixed tocopherols, preferably about 10 mg of mixed tocopherols. Ranges intermediate to the above-recited values, e.g., about 155 IU, 170 IU, 180 IU, etc., are also intended to be encompassed by the present invention.

Preferably, the supplements contain approximately 1.0 g of a n-6 fatty acid-containing oil (e.g., flaxseed and/or GLA-rich oils) combined with the appropriate amount of an n-3 rich oil rich in EPA and DHA to achieve the approximately 150-550 mg of EPA and approximately 50-500 mg of DHA e.g., a blend of a high EPA, i.e, 4510 (45% EPA and 10% DHA), and a high DHA, i.e., 1050 (10% EPA and 50% DHA), fish oil, and approximately 200 IU of vitamin E. Pre-made oil blends, such as a 30:20 blend (EPA:DHA), can also be used. Optionally, the supplements can further include 10-20 mg of mixed tocopherols, preferably 10 mg of mixed tocopherols. The ratio of the n-6 containing oil to the n-3 rich oil can also vary. For example, the ratios of flaxseed oil and/or GLA-rich oil to n-3 rich oil range from about 25% to 75% (1 to 3) to about 75% to 25% (3 to 1). Ranges intermediate to the above-recited values, e.g., about 30% to 70%, about 60% to 40%, and about 50% to 50% are also intended to be encompassed by the present invention. In one embodiment, 1.4 g of the preferred blends of n-3 rich oils provides approximately 450 mg of EPA and approximately 350 mg of DHA. To make the supplements smaller and easier to swallow, this daily dose is preferably divided into two (2), four (4) or more softgel capsules.

For example, a single softgel capsule using a 4510 blend of oil rich in EPA and a 1050 blend of oil rich in DHA would be formulated by combining 221 mg of the 4510 oil blend and 131 mg of the 1050 oil blend to produce 112.95 mg EPA/softgel capsule (99.5 mg EPA from the 4510 oil blend +13.5 mg EPA from the 1050 oil blend) and 87.60 mg DHA/softgel capsule (65.50 mg DHA from the 1050 oil blend +22.1 mg DHA from the 4510 oil blend). A daily dosage of four (4) softgel capsules would be administered to achieve the preferred 450 mg dose of EPA and 350 mg dose of DHA. For patients also afflicted with autoimmune diseases, e.g., Sjögren's syndrome or rheumatoid arthritis, twice the preferred daily dosage is recommended.

When used in vivo for therapeutic purposes, the nutritional supplements of the invention can be administered orally. Actual dosage levels of the active ingredients in the supplements of this invention may be varied so as to obtain an amount of the active ingredient which is effective to achieve the desired therapeutic response for a particular patient, i.e., a reduction in the symptoms associated with dry eye. The selected dosage level will depend upon a variety of pharmacokinetic factors including the activity of the particular supplements of the present invention employed, the time of administration, the rate of excretion of the particular compound being employed, the duration of the treatment, other drugs, compounds and/or materials used in combination with the particular compositions employed, the age, sex, weight, condition, diet, general health, the severity of dry eye, and conditions such as posterior blepharitis or meibomianitis, or meibomian gland dysfunction, and prior medical history of the patient being treated, and like factors well known in the medical arts. A physician or veterinarian having ordinary skill in the art can readily determine and prescribe the effective amount of the supplements required.

Accordingly, the present invention encompasses methods for treating a patient suffering from dry eye, meibomian gland inflammation, meibomian gland dysfunction or dry mouth by orally administering the nutritional supplements. In a preferred embodiment, the daily dose of the supplements are administered once in the morning or twice daily.

### EXAMPLES

### Example 1

### Formulation of a Nutritional Supplement for Treating Dry Eye

The nutritional supplements of the present invention can be formulated by mixing the following:

| | Amount of Daily | |
|---|---|---|
| | Dosage | |
| Vitamin E (as alpha-tocopherol concentrate) | 200 | IU |
| Mixed tocopherols | 10 | mg |
| Organic Flaxseed Oil | 1.0 | g |
| EPA (from fish oil)¹ | 450 | mg |
| DHA (from fish oil)¹ | 350 | mg |
| Other ingredients: gelatin; | | |
| glycerin; water; caramel color | | |

¹The EPA and DHA are added in the form of 1.4 g of a blend of fish oils rich in EPA and DHA.

The flaxseed oil is preferably organic (pesticide and herbicide free), cold-pressed to maintain the integrity of the alpha-linolenic oil (ALA). The high EPA and DHA fish oil may be a concentrated fish oil or any oil from a cold water fish species such as menhaden oil if it provides the proper amounts of EPA and DHA. The fish oil is preferably pharmaceutical grade (processed under nitrogen to prevent oxidation of the oils) and molecularly distilled to remove PCBs and other toxic substances. DHA may also be provided by marine algae. Vitamin E, or other oil soluble antioxidant, protects the integrity of the flaxseed oil and the EPA and DHA from oxidation. Vitamin E is also preferred in the supplements because if n-3 fatty acids are administered without vitamin E, the n-3 fatty acids in the serum deplete serum levels of vitamin E.

### Example 2

### Administration of a Nutritional Supplement for Treating Dry Eye

Case Study

FH, a 68 year old woman with dry eyes, was started on flaxseed oil at a dose of 1000 mg a day on Day 1. She returned on Day 60 and reported symptomatic improvement. At that time, 1000 mg of fish oil which was rich in EPA and DHA was added to her treatment regimen. By Day 120, she reported that the fish oil had “turbo-charged” or magnified the effect of the flaxseed oil treatment alone. It appears that the addition of the EPA and DHA-containing fish oil to the patient provides an unexpected effect of accelerating and improving the treatment of the dry eye.

### Example 3

### Dietary n-3 Fatty Acid Intake and Risk of Clinically Diagnosed Dry Eye Syndrome in Women

The relationship between dietary n-3 fatty acid intake and the risk of clinically diagnosed dry eye syndrome in women was examined. A total of 32,470 female health professionals aged between 45 and 84 years who provided information on diet and dry eye syndrome (DES) were chosen from the 39,876 women participating in the Women's Health Study. Intake of n-3 fatty acids was assessed by a validated food frequency questionnaire. DES was assessed using self-reports of clinically diagnosed DES. Logistic regression models to estimate the odds ratios (OR) and 95% confidence intervals (CI) to describe the relationships of n-3 fatty acid intake and DES was used. The relationship between consumption of fish and DES was also examined in a similar way.

After adjusting for age, other demographic factors, postmenopausal hormone therapy, and total fat intake, the OR (CI) for the highest versus the lowest dietary intake of n-3 fatty acids was 0.83 (0.70-0.98), P for trend=0.04. In addition, a significant association between tuna fish consumption and DES was observed (OR=0.82, CI=0.67-1.00 for 2 to 4 servings/week, and OR=0.34, CI=0.13-0.81 for 5 to 6 four-ounce servings/week versus <2 servings/week; P for trend=0.004). That is, an 18% reduction in dry eye syndrome was observed in patients who ate 2 to 4 servings of tuna versus those who ate less than 2 servings, and a 66% reduction in the diagnosis of dry eye in those who ate 5 to 6 servings a week, compared to those who ate less than 2 servings per week. Furthermore the dose response curve with increasing tuna doses was highly significant. These results were similar in other models additionally controlling for diabetes, hypertension, and connective tissue diseases.

These results show that women with a higher dietary intake of n-3 fatty acids are at decreased risk of developing DES. These findings are consistent with clinical observations and postulated biological mechanisms.

Incorporation by Reference

All patents, pending patent applications and other publications cited herein are hereby incorporated by reference in their entirety.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method for treating a condition selected from the group consisting of dry eye, meibomian gland inflammation meibomian gland dysfunction, and dry mouth comprising administering a nutritional supplement containing a n-6 fatty acid containing oil and a n-3 rich oil, wherein the n-3 rich oil contains a high concentration of eicosapentaenoic acid (EPA) and a high concentration of docosahexaeonic acid (DHA).

2. The method of claim 1, wherein the n-6 fatty acid containing oil further comprises a n-3 fatty acid.

3. The method of claim 2, wherein the n-6 fatty acid containing oil is flaxseed oil.

4. The method of claim 1, wherein the n-6 fatty acid containing oil is a GLA-rich oil.

5. The method of claim 4, wherein the n-6 fatty acid containing oil is selected from the group consisting of evening primrose oil, borage oil, and black currant seed oil.

6. The method of claim 3, further comprising an additional n-6 fatty acid, wherein the additional n-6 fatty acid is selected from the group consisting of evening primrose oil, borage oil, and black currant seed oil.

7. The method of claim 1, wherein a sufficient amount of the n-3 rich oil is administered to provide a daily dose of at least about 150-550 mg EPA and at least about 50-500 mg DHA.

8. The method of claim 1, wherein a sufficient amount of the n-3 rich oil is administered to provide a daily dose of at least about 350-450 mg EPA.

9. The method of claim 1, wherein the daily dose of n-3 rich oil comprises at least about 250-350 mg DHA.

10. The method of claim 1, wherein the ratio by weight of the n-6 fatty acid containing oil to the n-3 rich oil is about 1 to 1.

11. The method of claim 1, wherein the ratio by weight of the n-6 fatty acid containing oil to the n-3 rich oil is about 1 to 1.4.

12. The method of claim 1, wherein the ratio by weight of the n-6 fatty acid containing oil to the n-3 rich oil is about 1 to 1.5.

13. The method of claim 1, wherein the ratio by weight of the n-6 fatty acid containing oil to the n-3 rich oil is about 1 to 3.

14. The method of claim 1, wherein the ratio by weight of the n-6 fatty acid containing oil to the n-3 rich oil is about 3 to 1.

15. The method of claim 1, wherein the supplement further comprises an oil soluble antioxidant.

16. The method of claim 15, wherein the supplement further comprises d-alpha-tocopherol.

17. The method of claim 15, wherein the antioxidant is vitamin E.

18. The method of claim 17, wherein the vitamin E is d-alpha tocopherol.

19. The method of claim 17, wherein the daily dose of vitamin E is at least about 100-400 IU.

20. The method of claim 17, wherein the daily dose of vitamin E is at least about 200 IU.

21. The method of claim 15, wherein the antioxidant comprises about 5-10 mg of mixed tocopherols per daily dose.

22. The method of claim 1 wherein the nutritional supplement is administered orally.

23. The method of claim 22 wherein the nutritional supplement is administered as four (4) softgel capsules daily.

24. The method of claim 1, wherein the supplement comprises 1.0 g of a n-6 fatty acid containing oil, 1.4 g of a n-3 rich oil that provides approximately 450 mg of EPA and approximately 350 mg of DHA, approximately 200 IU of vitamin E, and approximately 10 mg of mixed tocopherols per daily dose.

25. The method of claim 1, wherein the supplement comprises 1.0 g of a n-6 fatty acid containing oil, 1.5 g of a n-3 rich oil that provides approximately 450 mg of EPA and approximately 300 mg of DHA, approximately 200 IU of vitamin E, and approximately 10 mg of mixed tocopherols per daily dose.

26. The method of claim 1, wherein the supplement comprises approximately 1.0 g of a n-6 fatty acid containing oil, approximately 1.4 g a n-3 rich oil that provides approximately 450 mg of EPA and approximately 350 mg of DHA, approximately 200 IU of vitamin E, and approximately 10 mg of mixed tocopherols, and wherein the supplement is administered in two doses daily.

27. The method of claim 1, wherein the supplement comprises approximately 1.0 g of a n-6 fatty acid containing oil, approximately 1.5 g a n-3 rich oil that provides approximately 450 mg of EPA and approximately 300 mg of DHA, approximately 200 IU of vitamin E, and approximately 10 mg of mixed tocopherols, and wherein the supplement is administered in two doses daily.

28. The method of claim 1, wherein the n-3 rich oil is administered in sufficient dosage to inhibit conversion of dihomo-gamma-linolenic acid (DGLA) to arachidonic acid (AA).

29. The method of claim 1, wherein the n-3 rich oil is administered in sufficient dosage to increase the production of prostaglandin PGE₁.

30. The method of claim 1, wherein the n-3 rich oil is administered in sufficient dosage to inhibit apoptosis of the lacrimal gland and corneal and conjunctival epithelium.

31. The method of claim 1, wherein the n-3 rich oil is administered in sufficient dosage to inhibit apoptosis of the salivary gland.

32. The method of claim 1, wherein the n-3 rich oil is administered in sufficient dosage to block the gene expression of TNF-α.

33. A nutritional supplement for treating a condition selected from the group consisting of dry eye, meibomian gland inflammation and meibomian gland dysfunction, and dry mouth consisting essentially of a nutritionally sufficient amount of a n-6 fatty acid containing oil, a therapeutic amount of a n-3 rich oil that provides approximately 150-550 mg of EPA and approximately 50-500 mg of DHA, approximately 150-250 IU of vitamin E, and approximately 5-20 mg of mixed tocopherols per daily dose.

34. A nutritional supplement for treating dry eye, meibomian gland inflammation, meibomian gland dysfunction or dry mouth consisting essentially of approximately 1.0 g of a n-6 fatty acid containing oil, approximately 1.4 g of a n-3 rich oil that provides approximately 450 mg of EPA and approximately 350 mg of DHA, approximately 200 IU of vitamin E, and approximately 10 mg of mixed tocopherols.

35. A nutritional supplement for treating dry eye, meibomian gland inflammation, meibomian gland dysfunction or dry mouth consisting essentially of approximately 1.0 g of a n-6 fatty acid containing oil, approximately 1.5 g of a n-3 rich oil that provides approximately 450 mg of EPA and approximately 300 mg of DHA, approximately 200 IU of vitamin E, and approximately 10 mg of mixed tocopherols.

36. The nutritional supplement of claim 33, wherein the ratio of the n-6 fatty acid containing oil to the n-3 rich oil is about 1 to 3.

37. The nutritional supplement of claim 33, wherein the ratio of the n-6 fatty acid containing oil to the n-3 rich oil is about 3 to 1.

38. A method of manufacturing a medicament for the treatment of a condition selected from the group consisting of dry eye, meibomian gland inflammation, meibomian gland dysfunction, and dry mouth whereby said medicament comprises a nutritionally sufficient amount of a n-6 fatty acid containing oil, a therapeutically effective amount of a n-3 rich oil that provides approximately 150-550 mg of EPA and approximately 50-500 mg of DHA, approximately 150-250 IU of vitamin E, and approximately 5-20 mg of mixed tocopherols.

39. The method of claim 38, wherein the medicament comprises approximately 1.0 g of a n-6 fatty acid containing oil, approximately 1.4 g of a n-3 rich oil that provides approximately 450 mg of EPA and approximately 350 mg of DHA, approximately 200 IU of vitamin E, and approximately 10 mg of mixed tocopherols.

40. The method of claim 38, wherein the medicament comprises approximately 1.0 g of a n-6 fatty acid containing oil, approximately 1.5 g of a n-3 rich oil that provides approximately 450 mg of EPA and approximately 300 mg of DHA, approximately 200 IU of vitamin E, and approximately 10 mg of mixed tocopherols.

41. The method of claim 36, wherein the ratio of the n-6 fatty acid containing oil to the n-3 rich oil is about 1 to 3.

42. The method of claim 36, wherein the ratio of the n-6 fatty acid containing oil to the n-3 rich oil is about 3 to 1.
